(19) United States
(12) Patent Application Publication
(10) Pub. No.: US 2008/0207593 A1
(43) Pub. Date: Aug. 28, 2008

(54) Antihistamine Combination

(75) Inventors: Michael Heffernan, Hingham, MA (US); Jane C. Hirsh, Wellesley, MA (US); Roman V. Rariy, Allston, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Collegium Pharmaceutical, Inc.

(21) Appl. No.: 11/841,438

(22) Filed: Aug. 20, 2007

Related U.S. Application Data
(60) Provisional application No. 60/892,189, filed on Feb. 28, 2007.

(51) Int. Cl. A61K 31/4545; A61K 31/135; A61K 31/40; A61K 31/4402; A61K 31/4439; A61K 31/506; A61K 31/55; A61P 37/08; A61P 17/00; A61K 31/5415; A61K 31/495; A61K 31/45; A61K 31/451; A61K 31/4535

(52) U.S. Cl. 514/214.02; 514/226.2; 514/255.04; 514/269; 514/290; 514/317; 514/324; 514/325; 514/327; 514/343; 514/352; 514/357; 514/428; 514/648

(57) ABSTRACT

A once daily formulation containing the combination of a non-sedating or selective antihistamine (Loratadine) with a sedating or non-selective antihistamine (Chlorpheniramine) administered at bed-time has demonstrated greater efficacy in relieving allergic symptoms than Loratadine alone.

### CROSS-REFERENCE TO RELATED APPLICATIONS

This application claims priority to U.S. Ser. No. 60/892,189, filed Feb. 28. 2007.

### BACKGROUND OF THE INVENTION

This technology is generally in the field of extended relief combination antihistamine formulations and treatment

Allergic rhinitis affects 10% to 30% of US adults and up to 40% of US children, resulting in estimated direct and indirect costs of $5.3 billion per year. Treatment includes avoidance of known allergens, pharmacotherapy, and, if necessary, immunotherapy. Most patients with allergic rhinitis experience early morning symptoms, either during the night or immediately upon awakening in the morning. Many patients describe explosive sneezing, nasal itching and runny nose upon awakening. Nocturnal symptoms of rhinitis are unpleasant and cause interference with sleeping, leading to significant sleep abnormalities. These symptoms are still a frequent problem in spite of the current therapeutic modalities for allergic rhinitis (Storms, W. J. Aller. Clin Immunol. 2004; 114(suppl):5).

Many single entity medications taken in the morning wear off at the end of their dosing cycle, leaving patients vulnerable for these early morning symptoms. Most of the older antihistamines are given in multiple daily doses (i.e. three to six times per day) and wear off 4, 8 or 12 hours after administration. In addition, most presently available over-the-counter remedies, such as chlorpheniramine or diphenhydramine, when given throughout the day, may cause troublesome side effects such as drowsiness, fatigue, and dry mouth. As an example of the clinical problems seen with once-a-day medications taken in the morning, loratadine is often ineffective in controlling nocturnal and early morning symptoms.

First generation, non-selective “sedating” antihistamines have been shown to be efficacious throughout a 24 hour dosing period when given at the approved dosage and frequency but lack an acceptable safety profile. In comparison, second generation, selective antihistamines offer a relatively good safety profile but often fail to provide the efficacy of non-sedating antihistamines throughout the 24 hour dosing period.

For example, a non-selective antihistamine, Chlorpheniramine, was studied by Kemp et al Ann Allergy. 1985 June; 54(6):502-509), who randomized 397 patients with seasonal allergic rhinitis to receive terfenadine 60 mg bid, chlorpheniramine 4 mg three times per day (12 mg daily) or placebo for seven days. Moderate or complete relief was reported by 60% of patients treated with chlorpheniramine but 19% reported sedation.

In contrast, Loratadine is an antihistamine agent exhibiting partial selectivity for peripheral histamine H1-receptors. To date, loratadine has been evaluated in allergic rhinitis, urticaria and, to a limited extent, in asthma. In several large controlled comparative clinical studies, loratadine was superior to placebo, faster acting than astemizole and as effective as azatadine, cetirizine, chlorpheniramine (chlorphenamine), clemastine, hydroxyzine, mequitazine and terfenadine in patients with allergic rhinitis and chronic urticaria. At dosages of 10 mg daily, sedation occurred less frequently with loratadine than with azatadine, cetirizine, chlorpheniramine, clemastine and mequitazine. (Haria et al. Drugs. 1994 October; 48(4):617-637). In the development of loratadine, optimal 24 hour efficacy was demonstrated with once daily doses of 20 and 40 mg, However, these doses were associated with significant sedation (34.3% of patients receiving loratadine 40 mg). Treatment with loratadine 10 mg once a day is associated with a lack of 24 hour efficacy. Ratner et al J. Aller. Clin. Immunol. 2000 June; 105(6 Pt 1):1101-1107). Van Adelsberg et al Allergy. 2003 December; 58(12):1268-1276) randomized 1,079 patients with seasonal allergic rhinitis to receive treatment with loratadine 10 mg QD, montelukast 10 mg QD or placebo for four weeks. Loratadine treatment was associated with a statistically significant reduction in symptom scores for the first two week period and the four week study overall. However, patients treated with loratadine did not show any improvement in nocturnal symptoms.

There are only few combination treatments known in the art. Mild, intermittent allergic rhinitis is most commonly treated with second-generation oral antihistamines such as loratadine or a combination of second generation oral antihistamine with a decongestant. Azelastine, a second-generation antihistamine, is available as a nasal spray. Recently, concomitant use of montelukast (a Leukotriene Receptor Antagonist) and loratadine as treatment for seasonal allergic rhinitis was investigated (Nayak, et al. Ann Allergy Asthma Immunol, 88(6), 592-600). It was found that the effect of montelukcast/loratadine combination compared with loratadine alone, the primary comparison, was not significantly different. The leukotriene receptor antagonists are selective and competitive antagonists of the cysteinyl leukotriene (Cys LT1) receptor, Cysteinyl leukotriene (LTC4, LTD4 and LTE4) production and receptor occupation have been correlated with the pathophysiology of asthma, including airway edema, smooth muscle constriction, and altered cellular activity associated with the inflammatory process.

Hence, the problem to be addressed is that physicians and patients are presently confined to a choice between poor efficacy in managing nocturnal symptoms by second generation non-sedating antihistamines and the poor side effect profile (i.e., daytime sedation) associated with first generation sedating antihistamines. Thus, there is a significant need for improved allergic rhinitis treatment providing greater efficacy throughout the entire day with fewer or diminished treatment related side effects.

U.S. patent application 20050069580 by Collegium Pharmaceutical describes combination formulations of selective and non-selective antihistamines to provide long term, once a day treatments. These typically consist of an immediate release (“IR”) formulation of one of the antihistamines, such as the non-sedating selective antihistamines, for a formulation to be taken in the morning, in combination with a delayed release (“DR”) formulation that releases a sedating or non-selective antihistamine late afternoon, or vice versa.

It is therefore an object of the present invention to provide antihistamine combinations that demonstrate improved efficacy and a non-inferior side effect profile when compared to individual therapeutic agents of the combination.

It is therefore a further object of the present invention to provide once a day antihistamine combinations that effectively alleviate the symptoms of allergic rhinitis for a 24 hour period including when the symptoms are typically worse, (i.e. night time and upon morning wakening) and do not increase the day time side effects including sedation.

### SUMMARY OF THE INVENTION

The combination of a second generation once daily selective antihistamine (Loratadine) with a first generation non-selective antihistamine (Chlorpheniramine) administered together at bed-time has demonstrated significant efficacy in relieving allergic symptoms. In the preferred embodiment, one of the antihistamines is provided as a delayed release formulation. In the most preferred embodiment, the non-selective antihistamine is provided as an immediate release formulation, most preferably as an outer coating around a core of a delayed release formulation of the selective antihistamine.

The combination of 4 mg Chlorpheniramine maleate, a non-selective antihistamine, in an IR formulation, and 10 mg Loratadine, a selective antihistamine, in a IR formulation, was tested in a clinical study. Within the limitations of a small study with relatively low, environmentally-derived (uncontrolled) pollen challenge, it is seen that the chlorpheniramine/loratadine combination, when given at bed time, outperforms loratadine alone administered at the same time, as well as placebo. It is important to note that the CHL/LOR combination better controls allergy symptoms both at AM and PM, as indicated by Instantaneous and Reflective TNSS scores. The fact that the combination of loratadine and chlorpheniramine outperforms loratadine alone 24 hours after administration (PM TNSS scores) is especially surprising, given that chlorpheniramine alone needs to be administered every 4 to 6 hours in order to be therapeutically effective.

### BRIEF DESCRIPTION OF THE DRAWINGS

FIG. 1 is a graph of the morning reflective TNSS score for Chlorpheniramine/loratadine, loratadine, and placebo when given at bed time.

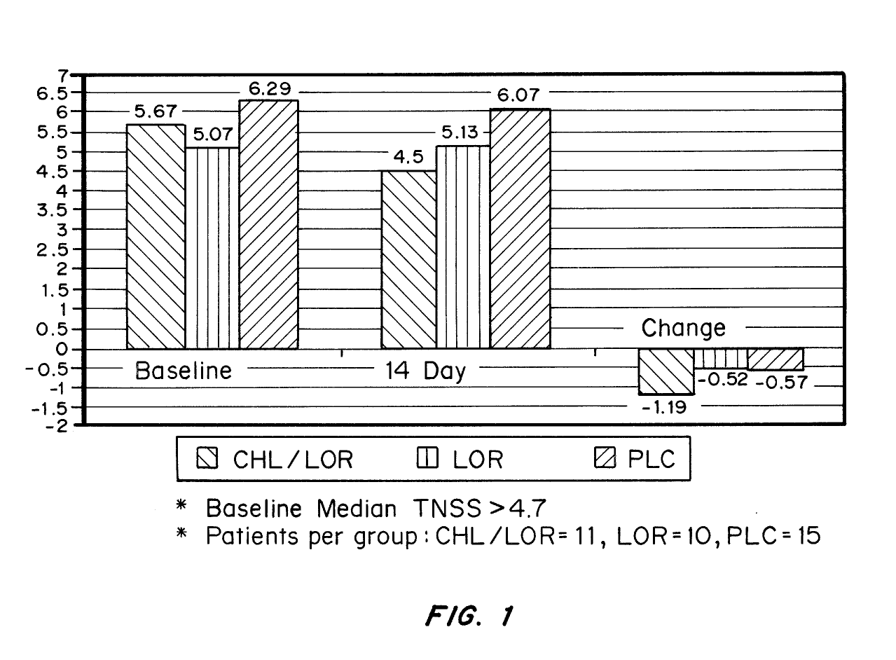

FIG. 2 is a graph of the evening reflective TNSS score for Chlorpheniramine/loratadine, loratadine, and placebo when given at bed time.

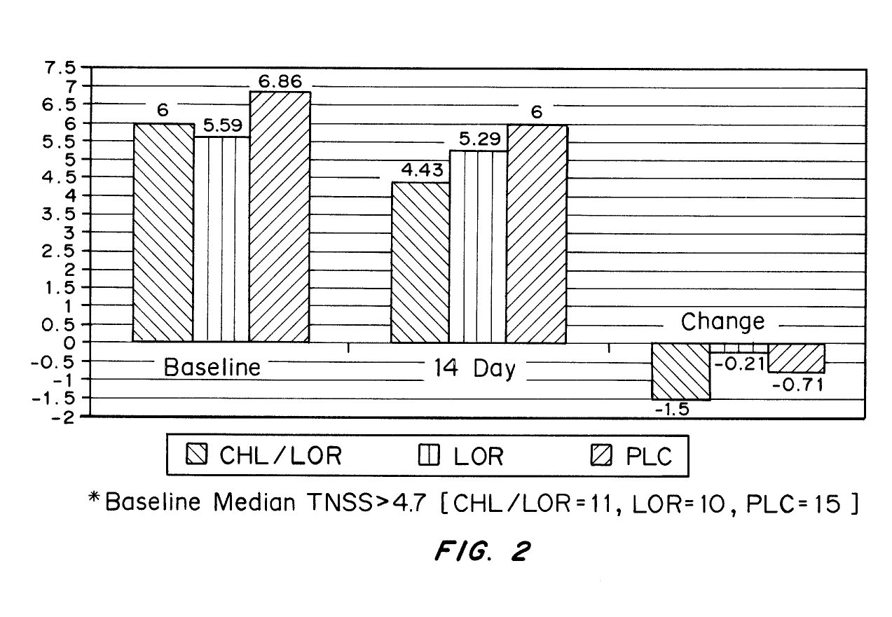

### DETAILED DESCRIPTION OF THE INVENTION

### I. Formulations

A. Definitions

As used herein, “sedating” antihistamines refer to older or first generation histamine compounds. The older antihistamines (first generation antihistamines) are associated with troublesome sedative and anti-muscarinic effects and are often called “sedating antihistamines” or “first generation antihistamines”.

As used herein, “Non-sedating antihistamines” refer to second generation or newer antihistamine compounds. These newer “second generation antihistamines” are essentially devoid of the sedative effect, and are often called “non-sedating antihistamines.”

As used herein, “Efficacious” and “effective” are synonymous, and mean having the power to produce a desired effect.

As used herein, “PM” is generally synonymous herein with “bedtime” or normal hour of going to bed.

As used herein, “Co-administration” is defined as administering antihistamine-containing formulations at essentially the same time in the same or different dosage forms.

As used herein, the term “antihistamine” is generally applied to Histamine H₁ receptor antagonists.

B. Antihistamines

Many sedating antihistamines are widely used and are available from the over the counter “OTC” market. Typical first generation antihistamines include brompheniramine, chlorpheniramine, dexbrompheniramine, dexchlorpheniramine, carbinoxamine, clemastine, diphenhydrarmine, pyrilamine, tripelennamine, tripolidine, methdilazine, bromodiphenhydramine, promethazine, azatadine, cyproheptadine, diphenylpyraline, doxylamine, trimeprazine, phenindamine, and hydroxyzine.

The sedative effect of the sedating antihistamines can range from slight drowsiness to deep sleep. Daytime sedation can be a problem especially for those who drive or who operate machinery.

Second generation antihistamines include evocetrizine dihydrochloride, fexofenadine, loratadine, descarboethoxyloratadine, norastemizole, desmethylastemizole, cetirizine, acrivastine, ketotifen, temelastine, ebastine, epinastine, mizolastine, and setastine. Cetirizine, in spite of being a second generation antihistamine, has a low to moderate sedative effect.

The combination of 4 mg Chlorpheniramine maleate and 10 mg Loratadine was used in the following non-limiting examples. The preferred Chlorpheniramine maleate dose range is from 2 to 70 mg a day and the most preferred range is between 4 and 24 mg a day. The preferred dose range for Loratadine is between 2 and 80 mg, with the most preferred range being between 5 and 40 mg per day.

B. Carriers

As used herein, a modified release dosage form is one for which the drug release characteristics of time course and/or location are chosen to accomplish therapeutic or convenience objectives not offered by conventional dosage forms such as solutions, ointments, or promptly dissolving dosage forms, such as effervescent formulations or immediate release tablets. Delayed release, extended release, and pulsatile release dosage forms and their combinations are types of modified release dosage forms.

As used herein, a delayed release dosage form is one that releases a drug (or drugs) at a time other than promptly after administration.

As used herein, an extended release dosage form is one that allows at least a twofold reduction in dosing frequency as compared to that drug presented as a conventional dosage form (e.g. as a solution or prompt drug-releasing, conventional solid dosage form).

As used herein, a pulsatile release dosage form is one that mimics a multiple dosing profile spaced in time (for example, twice daily “BID”, three times daily “TID”) without repeated administration, and allows at least a twofold reduction in dosing frequency as compared to the drug presented as a conventional dosage form (e.g. as a solution or prompt drug-releasing, conventional solid dosage form).

As used herein, the term “taste masking coating” refers to a pH dependent coating that is insoluble in the mouth but dissolves in the acidic pH of the stomach.

As used herein, the term “extended release coating” refers to a pH independent substance that will act as a barrier to control the diffusion of the drug from its core complex into the gastrointestinal fluids.

As used herein, the term “enteric coating” refers to a coating material which remains substantially intact in the acid environment of the stomach, but which dissolves in the environment of the intestines.

As used herein the term “delayed release coating” refers to a pH dependent coating that is insoluble in the acidic pH of the stomach, the pH within the upper small intestine, but dissolves within the lower small intestine or upper large intestine.

Formulations are prepared using a pharmaceutically acceptable “carrier” composed of materials that are considered safe and effective and may be administered to an individual without causing undesirable biological side effects or unwanted interactions. As used herein, the “carrier” is all components present in the pharmaceutical formulation other than the active ingredient or ingredients. The term “carrier” includes but is not limited to diluents, binders, lubricants, desintegrators, fillers, and coating compositions. “Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et.al., (Media, Pa.: Williams and Wilkins, 1995) which provides information on carriers, materials, equipment and process for preparing tablets and capsules and delayed release dosage forms of tablets, capsules, and granules.

Suitable formulations and methods of manufacture can be found in U.S. Pat. Nos. 6,863,901 and 6,827,946 and US.S.N. 20050123609 and 20050069580.

1. Solid Formulations

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pre-gelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydrorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. The anionic or amphoteric surfactants may be present as pharmaceutically accepatable salts, including for example sodium, potassium, ammonium salts. Examples of anionic surfactants include long chain alkyl sulfonates and alkyl aryl sulfonates such as sodium dodecylbenzene sulfonate; dialkyl sodium sulfosuccinates, such as sodium bis-(2-ethylthioxyl)-sulfosuccinate; and alkyl sulfates such as sodium lauryl sulfate.

Cationic surfactants include, but are not limited to, quaternary ammonium compounds such as benzalkonium chloride, benzethonium chloride, cetrimonium bromide, stearyl dimethylbenzyl ammonium chloride, and cocoamine. Examples of nonionic surfactants include polyoxyethylene, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, poloxamer 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### Extended Release Dosage Forms

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and Carbopol® 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### Delayed Release Dosage Forms

Delayed release formulations can be created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers. These may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, or fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### Ion-Exchange Resins as Complexing Agents

Drug complexes are generally prepared by complexing the drug with a pharmaceutically acceptable ion-exchange resin. The complex is formed by reaction of a functional group of the drug with a functional group on the ion exchange resin. For antihistamine, the basic amino group can complex with an ion-exchange resin that bears an acidic group such as a sulfate or carboxylate group. Drug is released by exchanging with appropriately charged ions within the gastrointestinal tract.

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be modified by the addition of ionic groups. The covalently bound salt forming groups may be strongly acidic (e.g., sulfonic acid or sulfuric acid) or weakly acidic (e.g., carboxylic acid). In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975), incorporated by reference herein.

Resins suitable for use in the present invention include, but are not limited to Amberlite IRP-69 (Rohm and Haas) INDION 224, INDION 244, and INDION 254 (Ion Exchange (India) Ltd.). These resins are sulfonated polymers composed of polystyrene cross-linked with divinylbenzene. Any ion-exchange resins currently available and those that should become pharmaceutically acceptable and available in the future can also be used. Commercial sources of ion exchange resins that are either pharmaceutically acceptable or may become pharmaceutically acceptable in the future include, but are not limited to, Rohm and Haas, The Dow Chemical Company, and Ion Exchange (India) Ltd.

The size of the ion-exchange particles should be less than about 2 millimeter, more preferably below about 1000 micron, more preferably below about 500 micron, and most preferably below about 150 micron. Commercially available ion-exchange resins (Amberlite IRP-69, INDION 244 and INDION 254) have a particle size range less than 150 microns.

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778, 4,894,239, and 4,996,047.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

Antihistamine-containing resin particles can be coated with taste-masking coating. Taste-masking coating prevents the release of drug within the mouth and insures that no unpleasant, biter taste is experienced by the patient consuming the dosage form.

The cationic polymer Eudragit® ED 100 (Rohm Pharma) carries amino groups. Its films are, therefore, insoluble in the neutral medium of saliva, but dissolve by salt formation in the acid environment of the stomach. Such film coatings with a thickness of approximately 10 micrometers prevent medication with a bitter or revolting taste from dissolving in the mouth upon ingestion or during swallowing. The protective film dissolves quickly in the stomach allowing for the active ingredient to be released. A sugar coating may be used to accomplish similar taste-masking effect, albeit coating must be over 100 times thicker and these larger particles may result in tickling or irritating the throat.

In some embodiments drug-resin complexes are coated with a pH sensitive polymer which is insoluble in the acid environment of the stomach, and soluble in the more basic environment of the GI tract. The outer coating is thus an enteric coating; such dosage form is designed to prevent drug release in the stomach. Preventing drug release in the stomach has the advantage of reducing side effects associated with irritation of the gastric mucosa. Avoiding release within the stomach can be achieved using enteric coatings known in the art. The enteric coated formulation remains intact or substantially intact in the stomach, however, once the formulation reaches the small intestines, the enteric coating dissolves and exposes either drug-containing ion-exchange resin particles or drug-containing ion-exchange resin particles coated with extended release coating.

The enteric coated particles can be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et.al., (Media, Pa.: Williams and Wilkins, 1995). Examples of suitable coating materials include but are not limited to cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Rohm Pharma). Additionally the coating material may contain conventional carriers such as plasticizers, pigments, colorants, glidants, stabilization agents, and surfactants.

Extended release pharmaceutical compositions are obtained by complexing antihistamine with a pharmaceutically acceptable ion-exchange resin and coating such complexes with a substance that will act as a barrier to control the diffusion of the drug from its core complex into the gastrointestinal fluids.

Control of the release of drugs from drug-resin complexes is possible with the use of a diffusion barrier coating on the drug-resin complex particles. Several processing methods to achieve extended release coatings on drug loaded resin particles have been described (see, for example, U.S. Pat. Nos. 4,996,047, 4,221,778, and 4,894,239); any of these may be used to obtain the extended release antihistamine composition. Extended release coated antihistamine-resin complexes can also be prepared without the use of impregnating agents.

In general, any coating procedure which provides a contiguous coating on each particle of drug-resin complex without significant agglomeration of particles may be used. Coating procedures known in the pharmaceutical art including, but not limited to, fluid bed coating processes and microencapsulation may be used to obtain appropriate coatings. The coating materials may be any of a large number of natural or synthetic film-formers used singly, in admixture with each other, and in admixture with plasticizers (for example, Durlcex 500 vegetable oil), pigments and other substances to alter the characteristics of the coating. In general, the major components of the coating should be insoluble in, and permeable to, water. However, it might be desirable to incorporate a water-soluble substance, such as methyl cellulose, to alter the permeability of the coating. The coating materials may be applied as a suspension in an aqueous fluid or as a solution in organic solvents. The water-permeable diffusion barrier may consist of ethyl cellulose, methyl cellulose and mixtures thereof. The water-permeable diffusion barrier may also consist of water insoluble synthetic polymers sold under the trade name Eudragit® (Rohm Pharma), such as Eudragit RS, Eudragit RL, Eudragit NE and mixtures thereof. Other examples of such coating materials can be found in the Handbook of Pharmaceutical Excipients, Ed. By A. Wade and P. J. Weller, (1994).

As used herein, the term water-permeable is used to indicate that the fluids of the alimentary canal will permeate or penetrate the coating film with or without dissolving the film or parts of the film. Depending on the permeability or solubility of the chosen coating (polymer or polymer mixture) a lighter or heavier application thereof is required to obtain the desired release rate.

U.S. Pat. No. 4,221,778 to Raghunathan describes the addition of solvating agents such as polyethylene glycol to the system in order to reduce the swelling of the drug-loaded resins and prevent the fracturing of the extended release coating. The solvating agent can be added as an ingredient in the resin drug complexation step or preferably, the particles can be treated with the solvating agent after complexing. This treatment has not only been found to help the particles retain their geometry, but has enabled the effective application of diffusion barrier coatings such as ethylcellulose to such particles. Other effective solvating (impregnating) agent candidates include, for example, propylene glycol, glycerin, mannitol, lactose and methylcellulose. Up to about 30 parts by weight (normally 10-25 parts) of the solvating agent to 100 parts by weight of the resin has been found to be effective. EP 171,528, EP 254,811, and EP 254,822 all disclose similar impregnation treatments in order to improve coatability of resin complexes.

Control of the release of drugs from drug-resin complexes has been achieved by the direct application of an ethylcellulose diffusion barrier coating to particles of such complexes in the absence of an impregnating agent, provided that the drug content of the complexes was above a critical value. U.S. Pat. No. 4,996,047 to Kelleher et al., discloses extended release coated drug-resin complexes wherein the drug comprises more than about 38% by weight (for irregularly shaped particles) of the dry drug-resin complex (based on the free acid or base of drug). In order to achieve this relatively high loading, a method of complexing drug to resin is provided whereby the drug is combined in its basic form with the resin in its acidic form (or visa versa). Since no ionic by-products are formed in such a reaction, very high loading levels are achieved. A similar scheme was disclosed in U.S. Pat. No. 4,894,239 to Nonomura, et al, with the free form of the drug being formed as part of a continuous process. U.S. Pat. No. 4,894,239 states the drug-resin complex should contain at least 80% of the theoretical ion adsorption amount, and more preferably should contain about 85 to 100% of theoretical ion adsorption amount, to produce a stable coating on the final drug-resin complex.

U.S. Pat. No. 5,186,930, Kogan et al. discloses drug-resin particles coated with a first inner coating of wax and a second outer coating of a polymer to achieve extended release. The inner wax coating prevents the swelling of the resins and subsequent rupturing of the extended release polymer coating.

In addition to known methods of processing drug-loaded resins to obtain stable extended release coatings, it was found that coating of antihistamine loaded ion-exchange resins with an acrylic polymer based coating (Eudragit® RS) results in a stable extended release composition without use of impregnating agents even when the drug loading is conducted by binding the salt form of the drug with the salt form of the resin, rather than binding the free base of the drug with resin in its acidic form as described by Kelleher et al and Nonomura et al. Antihistamine-resin complexes obtained by binding the salt form of the drug with the salt form of the resin have drug loadings lower than Kelleher et al and Nonomura et al reported as necessary to obtain stable extended release coatings without the use of impregnating agents.

In some embodiments drug-resin complexes are coated with a pH sensitive polymer which is insoluble in the acid environment of the stomach, insoluble in the environment of the small intestines, and soluble in the conditions within the lower small intestine or upper large intestine (e.g., above pH 7.0). Such a delayed release form is designed to prevent drug release in the upper part of the gastrointestinal (GI) tract.

The delayed release particles can be prepared by coating drug-containing microparticles with a selected coating material, as described above for delayed release coatings in general.

Delayed release coated particles can be administered simultaneously with an immediate release dose of the drug. Such a combination produces the modified release profile referred to as “pulsatile release”. By “pulsatile” is meant that drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Optionally, a second pulse is followed by a second time interval (lag time) during which very little or no drug is released from the dosage form, after which a third dose is then released.

The first pulse of the pulsatile release composition can be obtained by administering unmodified drug, uncoated drug-resin particles, taste-masked coated drug-resin particles, or, in some cases, enteric coated drug-resin particles along with delayed release coated particles that provide a second pulse.

In some cases it may be advantageous to combine an immediately releasing dose of drug (eg, unmodified drug, uncoated drug-resin particles, or taste masking coated drug-resin particles) with enteric coated drug-resin particles to create a pulsatile profile. In this case the first pulse will occur substantially immediately and the second pulse will occur once the enteric coating has dissolved (in the upper small intestines).

In order to create a final dosage form with three pulses, an immediate release dose of drug (e.g., unmodified drug, uncoated drug-resin particles, or taste masking coated drug-resin particles) can be combined with enteric coated drug-resin particles and delayed release coated drug resin particles.

2. Liquid Formulations

Typically, the carrier in a liquid formulation will include water and/or ethanol, flavorings (bubblegum is a favorite for pediatric use) and colorings (red, orange, and purple are popular).

Coated drug-resin particles are suitable for suspending in an essentially aqueous vehicle with the only restrictions on its composition being (i) an absence of, or very low levels of ionic ingredients, and (ii) a limitation on the concentrations of water-miscible organic solvents, such as alcohol, and on the pH, to those levels which do not cause dissolution of the diffusion barrier and enteric coatings. Liquid oral dosage forms include aqueous and nonaqueous solutions, emulsions, suspensions, and solutions and/or suspensions reconstituted from non-effervescent granules, containing suitable solvents, emulsifying agents, suspending agents, diluents, sweeteners, coloring agents, and flavoring agents. Preservatives may or may not be added to the liquid oral dosage forms. Specific examples of pharmaceutically acceptable carriers and excipients that may be used to formulate oral dosage forms are described in U.S. Pat. No. 3,903,297 to Robert.

In preparing the liquid oral dosage forms, the drug-resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. The most preferred carrier is a suspension of the pharmaceutical composition in an aqueous vehicle containing a suitable suspending agent. Suitable suspending agents include Avicel RC-591 (a microcrystalline cellulose/sodium carboxymethyl cellulose mixture available from FMC), guar gum and the like. Such suspending agents are well known to those skilled in the art.

Although water itself may make up the entire carrier, typical liquid formulations preferably contain a co-solvent, for example, propylene glycol, glycerin, sorbitol solution, to assist solubilization and incorporation of water-insoluble ingredients, such as flavoring oils into the composition.

3. Chewable, Crushable, or Rapidly Dissolving Tablets

In some embodiments coated drug-resin complexes are incorporated into chewable tablets, crushable tablets, or tablets which dissolve rapidly within the mouth. Chewable tablet formulations containing coated particles are known in the pharmaceutical arts (see for instance the textbook “Pharmaceutical dosage form—tablets” Vol. 1 edited by H A Lieberman et al. Marcel Dekker, Inc. (1989). Crushable tablets are conventional tablets that have the same in vitro and in vivo performance regardless of their physical integrity, i.e. tablets can be crushed and administered as a powder, e.g. on apple sauce, or mixed with water and syringed into a nasogastric or jejunostomy tube. The crushable tablets can be prepared using methods of tablet manufacturing known in the pharmaceutical art. Fast dissolving tablets containing coated particles are described, for example, in U.S. Pat. No. 6,596,311.

4. Gels

In some embodiments coated drug-resin complexes are incorporated into gels. Ion-exchange resin containing gel compositions are known in the art, see, for example, U.S. Pat. No. 4,837,255.

5. Reconstitutable Dosage Units

Coated drug-resin complexes can be formulated into a granular material and packaged in a sachet, capsule or other suitable packaging in unit dose. Such granular material can be reconstituted at the time of use into a suitable vehicle such as water. The granular material may contain excipients that facilitate the dispersion of the particles in water. Formulations of this type have been disclosed in U.S. Pat. No. 6,077,532.

Other optional ingredients well known to the pharmaceutical art may also be included in amounts generally known for these ingredients, for example, natural or artificial sweeteners, flavoring agents, colorants and the like to provide a palatable and pleasant looking final product, antioxidants, for example, butylated hydroxy anisole or butylated hydroxy toluene, and preservatives, for example, methyl or propyl paraben or sodium benzoate, to prolong and enhance shelf life.

### II. Methods of Administration

The non-selective first generation antihistamine is administered in combination with the selective second generation antihistamine. This can be provided as a blister pack or other two-component package, containing one pill of each type, where the person is instructed to take one of each at bed time; or, it can be provided as a two component tablet, capsule or liquid, wherein one component is the non-selective antihistamine and the other component is the selective antihistamine. The antihistamines can be packaged separately or mixed together, as in the case where beads of each drug are mixed together in a capsule. In a preferred embodiment, the formulation is sold with packaging material describing the advantage of administering the two types of antihistamine together at bedtime to maximize symptom relief while minimizing next day drowsiness.

Even though in the examples given below Immediate Release dosage forms of sedating and non-sedating antihistamines were co-administered, the release of either antihistamine could be altered to achieve the desired therapeutic effect. For example, Immediate Release formulations of sedating antihistamine can be combined with Extended, Pulsatile, Delayed or Delayed-Extended Release formulation of non-sedating antihistamine. In the preferred embodiment, the release of non-sedating antihistamine is delayed for one to eight hours after the dose is administered. In the most preferred embodiment, sedating antihistamine is available immediately and non-sedating antihistamine is released one to six hours after the dosage form administration.

Similarly, immediate release combination of non-sedating antihistamine can be combined with Extended, Pulsatile, Delayed or Delayed-Extended Release formulation of a sedating antihistamine.

In Example 1 below, the combination of 4 mg IR Chlorpheniramine maleate and 10 mg IR Loratadine was used. It is important to note that the daily dose of Chlorpheniramine maleate can differ from 4 mg and that the daily dose of Loratadine can differ from 10 mg. The preferred Chlorpheniramine maleate dose range is 2-70 mg a day and the most preferred range is 4-24 mg a day. The preferred dose range for Loratadine is 2-80 mg with the most preferred range being 5-40 mg per day.

Alternatively one or both of the antihistamines may be administered by an alternative route, for example, nasal or pulmonary, in solution or suspension, or one may be administered by one of these routes while the other is administered orally.

The present invention will be further understood by reference to the following non-limiting examples.

### III. Examples

In the tables and figures below, abbreviations include: PLC—placebo, LOR—Loratadine, CHL—Chlorpheniramine maleate, TNSS—Total Nasal Symptom Score.

### Example 1

### Proof of Concept Clinical Study

### 1.1 Study Design

An open-label, placebo-controlled parallel-group comparison study was conducted. The protocol used was a 1 week run-in period, with daily placebo (PLC) for all groups, and a second 2 week period for treatment. Study participants were randomized to receive at bedtime (10 pm) during the treatment period, one of the following: PLC only; Immediate Release LOR 10 mg, or Immediate Release CHL 4 mg/Immediate Release LOR 10 mg. Commercially available IR tablets of CHL and LOR were packaged into capsules and used in this study. Participants filled out patient diaries in the morning (8 am) and evening (10 pm, prior to dosing). Reflective and Instantaneous TNSSs were recorded daily. Pittsburgh Sleep Quality Index (PSQI) was scored at the end of the study.

2.1 TNSS (Total Nasal Symptom Score)

Each symptom (rhinorrhea, nasal itching, sneezing) was scored on the following scale (Max Score=9):

0=absent symptoms (no sign/symptom evident);

1=mild symptoms (signs or symptoms clearly present but with minimal awareness, and easily tolerated);

2=moderate symptoms (definite awareness of signs or symptoms that are bothersome but tolerable); or

3=Severe symptoms (signs or symptoms that are hard to tolerate and cause interference with activities of daily living and/or sleeping).

At each time point (at 8 AM and 10 PM), symptoms were scored for Instantaneous and Reflective scores.

2.2 Study Population

Inclusion criteria: male/female 18-60 years, history of seasonal allergic rhinitis for at least two years, sensitivity to ragweed or locally prevalent allergen, immunotherapy at stable dose, AM Reflective TNSS=3 on at least 5 out of 7 consecutive days during run-in.

Exclusion criteria: subjects with asthma other than mild intermittent asthma, use of antihistamines, other than study medication; use of systemic or topical corticosteroids, use of decongestants or antihistamines within 3 days of Visit 1, nasal cromolyn within 2 weeks of Visit 1, nasal or systemic corticosteroids within 28 days of Visit 1, loratadine within 10 days of Visit 1, history of sleep abnormalities, sinusitis, use of medications causing drowsiness/interfere with sleep, subjects with a known history of alcohol or drug abuse, and other standard exclusions.

2.3 Study Sites. The Study was conducted in Newport News, Va. (20 subjects), Richmond, Va. (20 subjects), New Braunfels, Tex. (25 subjects), San Antonio, Tex. (9 subjects).

2.4 Study Results
2.4.1 Adverse events

No serious adverse events or deaths were observed. No indication of safety difference between loratadine and combination was found. Low incidence of sedation was observed: N=2 (loratadine), N=0 (combination or placebo).
2.4.2 AM TNSS Reflective Score efficacy analysis for 50% greatest severity cases (Baseline Median TNSS>4.7)

TABLE 1
AM TNSS Reflective Score (TNSS > 4.7)

| | | Combination | Loratadine | Placebo |
|---|---|---|---|---|
| Visit | | (N = 11) | (N = 10) | (N = 15) |
| Baseline | N | 11 | 10 | 15 |
| | Mean | 5.82 | 5.91 | 6.42 |
| | Std Dev | 0.67 | 1.27 | 1.12 |
| | Median | 5.67 | 5.07 | 6.29 |
| | Min, Max | 5.00, 6.86 | 5.00, 8.14 | 4.80, 8.00 |
| Week 2 | N | 11 | 10 | 15 |
| | Mean | 4.70 | 5.08 | 5.82 |
| | Std Dev | 1.45 | 1.98 | 1.19 |
| | Median | 4.50 | 5.13 | 6.07 |
| | Min, Max | 2.86, 8.00 | 2.21, 7.43 | 3.60, 7.53 |
| Change+ | N | 11 | 10 | 15 |
| | Mean | −1.12 | −0.82 | −0.60 |
| | Std Dev | 1.42 | 1.51 | 0.89 |
| | Median | −1.19 | −0.52 | −0.57 |
| | Min, Max | −3.71, 1.14 | −2.79, 1.29 | −2.45, 0.90 |

2.4.3 PM TNSS Reflective Score efficacy analysis for 50% greatest severity cases (Baseline Median TNSS>4.7)

TABLE 2
PM TNSS Reflective Score (TNSS > 4.7)

| | | Combination | Loratadine | Placebo |
|---|---|---|---|---|
| Visit | | (N = 11) | (N = 10) | (N = 15) |
| Baseline | N | 11 | 10 | 15 |
| | Mean | 5.88 | 6.10 | 6.62 |
| | Std Dev | 0.67 | 1.28 | 1.42 |
| | Median | 6.00 | 5.59 | 6.86 |
| | Min, Max | 5.00, 7.00 | 4.57, 8.50 | 3.71, 8.33 |
| End-of- | N | 11 | 10 | 15 |
| Treatment | Mean | 4.78 | 5.18 | 6.12 |
| | Std Dev | 1.38 | 1.94 | 1.12 |
| | Median | 4.43 | 5.29 | 6.00 |
| | Min, Max | 3.07, 8.14 | 2.14, 7.71 | 4.14, 7.57 |
| Change+ | N | 11 | 10 | 15 |
| | Mean | −1.10 | −0.92 | −0.50 |
| | Std Dev | 1.54 | 1.71 | 1.30 |
| | Median | −1.50 | −0.21 | −0.71 |
| | Min, Max | −3.93, 1.43 | −3.45, 1.21 | −2.43, 2.00 |

2.4.4 AM TNSS Instantaneous Score efficacy analysis for 50% greatest severity cases (Baseline Median TNSS>4.7)

TABLE 3
AM TNSS Instantaneous Score (TNSS > 4.7)

| | | Combination | Loratadine | Placebo |
|---|---|---|---|---|
| Visit | | (N = 11) | (N = 10) | (N = 15) |
| Baseline | N | 11 | 10 | 15 |
| | Mean | 5.90 | 5.58 | 6.00 |
| | Std Dev | 1.21 | 1.32 | 1.30 |
| | Median | 5.86 | 5.43 | 6.14 |
| | Min, Max | 4.14, 8.63 | 3.71, 7.71 | 3.60, 8.00 |
| Week 2 | N | 11 | 10 | 15 |
| | Mean | 4.64 | 4.83 | 5.51 |
| | Std Dev | 1.76 | 1.96 | 1.39 |
| | Median | 4.62 | 5.16 | 5.71 |
| | Min, Max | 1.60, 7.86 | 2.14, 7.36 | 2.50, 7.80 |
| Change+ | N | 11 | 10 | 15 |
| | Mean | −1.26 | −0.74 | −0.49 |
| | Std Dev | 1.45 | 1.20 | 0.79 |
| | Median | −1.01 | −0.83 | −0.64 |
| | Min, Max | −3.79, 1.14 | −2.62, 1.21 | −1.70, 0.93 |

2.4.5 M TNSS Instantaneous Score efficacy analysis for 50% greatest severity cases (Baseline Median TNSS>4.7)

TABLE 4
PM TNSS Instantaneous Score (TNSS > 4.7)

| | | Combination | Loratadine | Placebo |
|---|---|---|---|---|
| Visit | | (N = 11) | (N = 10) | (N = 15) |
| Baseline | N | 11 | 10 | 15 |
| | Mean | 5.78 | 5.70 | 5.93 |
| | Std Dev | 1.35 | 1.43 | 1.32 |
| | Median | 5.50 | 5.21 | 5.86 |
| | Min, Max | 4.14, 8.86 | 3.86, 8.83 | 3.71, 8.33 |
| Week 2 | N | 11 | 10 | 15 |
| | Mean | 4.52 | 4.89 | 5.58 |
| | Std Dev | 1.68 | 1.95 | 1.23 |
| | Median | 4.46 | 5.32 | 5.71 |
| | Min, Max | 2.29, 8.00 | 2.15, 7.21 | 3.14, 7.19 |
| Change+ | N | 11 | 10 | 15 |
| | Mean | −1.26 | −0.81 | −0.35 |
| | Std Dev | 1.90 | 1.58 | 1.19 |
| | Median | −0.90 | −0.21 | −0.50 |
| | Min, Max | −4.31, 1.86 | −3.45, 1.00 | −2.11, 1.93 |

2.5 Study Discussion and Conclusions

The study demonstrates many of the challenges of allergic rhinitis studies. Low pollen count resulted in low baseline symptom scores, compared to most studies of this sort. When the most severely affected 50% of the population (TNSS>4.7) was selected for the analysis, it demonstrated consistent benefit in the morning and evening scores from treatment. No changes were seen on the PSQI in terms of ability to sleep.

Within the limitations of a small study with relatively low, environmentally-derived (uncontrolled) pollen challenge, it is seen that the chlorpheniramine/loratadine combination, when given at bed time, outperforms loratadine alone administered at the same time, as well as placebo. It is important to note that the CHL/LOR combination better controls allergy symptoms both at AM and PM, as indicated by Instantaneous and Reflective TNSS scores. The fact that the combination of loratadine and chlorpheniramine outperforms loratadine alone 24 hours after administration (PM TNSS scores) is especially surprising, given that chlorpheniramine alone needs to be administered every 4 to 6 hours in order to be therapeutically effective (see Federal Register, vol. 57, No. 237, page 58365).

In summary, IR Loratadine and IR Chlorpheniramine administered together demonstrated improved efficacy and a non-inferior side effect profile when compared to Loratadine alone and effectively alleviated the symptoms of allergic rhinitis for a 24 hour period including when the symptoms are typically worse, (i.e. night time and upon morning wakening).

### Example 2

### Preparation of Immediate Release Loratadine Core Tablet

Loratadine USP (Micronized) from Morpen Labs (India) was used to manufacture immediate release (IR) Loratadine tablets. Particle size distribution of Loratadine, as determined using a Malvern Mastersizer 2000, was as following: 10% particles below 5 microns, 50% particles below 10 microns and 90% particles below 20 microns. All % particles are measured in % volume.

A wet granulation process was used for granulation. This process consisted of the steps of dry blending, wet granulation, drying, size reduction and final blending with extra-granular disintegrate and lubricant.

The first step of tablet granulation process was sifting Loratadine®, starch, lactose monohydrate and Avicel® PH101 (Microcrystalline cellulose) through 30 mesh sieve (600 μm). The second step consisted of dry blending of sifted material in a planetary mixer at low shear. In a separate container, polyvinyl pyrrolidone (Kollidon® 30) and sodium lauryl sulfate were mixed in de-mineralized water. This solution was slowly added to a powder mix in planetary mixer while mixing with low shear for 5 min. Granules obtained from this process were dried in a fluid bed dryer, keeping product bed temperature at 36-38° C. with an inlet temperature of 60° C. Drying time was 2-3 hours. Obtained granules were sifted through 20 mesh (850 μM) and loaded into a cone blender. Aerosil®-200 (Colloidal silicon dioxide) and Kollidon® CL (Crospovidone®) sifted through 60 mesh (250 μM) were then added to granulation in cone blender and mixed for 15 min at 15-17 rpm. Finally, Magnesium stearate, which was sifted through 60 mesh (250 μM), was added to granulation blend in cone mixer and mixer for additional 2 min. at a speed of 15-17 rpm.

This batch size was 25,000 tablets, each containing 10 mg Loratadine. Tablet composition of Lot # 1 is given below:

TABLE 5
Tablet composition for Lot #1

| | Amount per | |
|---|---|---|
| Ingredients | tablet (mg) | |
| Intra-granular | | |
| Loratadine USP | 10 | |
| Starch | 10 | |
| Lactose Pharmatose 200M | 34.7 | |
| Avicel PH101 | 17 | |
| Kollidon 30 | 2.4 | |
| Sodium Lauryl sulfate | 2 | |
| DM water | q.s. | |
| Extra-granular | | |
| Aerosil-200 | 0.5 | |
| Kollidon CL | 2.4 | |
| Magnesium stearate | 1 | |
| Total tablet wt. | 80 | mg |

Tablets were compressed with 16 station single rotary compression machine with 6 mm circular concave punches and following tablet parameters were obtained. Tablets weight 80 mg, were 2.51-2.61 inches thick, had a diameter of 6 mm, a friability of 0.1% and a disintigration time of 4 min 50 sec.

### Example 3

### Alternate Loratadine Core Tablet

In this example, tablets were made using a dry granulation method. The first step of tablet manufacturing process was sifting micronized Loratadine, lactose, Prosolv® 50, Ac-Di-Sol®, and SDS through 40 mesh sieve (425 micron). The second step consisted of dry mixing of sifted material in V-cone blender for 20 minutes. Aerosil® and Magnesium stearate sifted through 40 mesh sieve were then added and the resultant blend further mixed for 10 minutes. The final blend was compressed into tablets using 6 mm round standard concave punch at a hardness of 30-50 N.

TABLE 6
Tablet composition for Loratadine IR tablets

| | Amount per |
|---|---|
| Ingredient | tablet (mg) |
| Loratadine USP | 10.00 |
| Lactose (Pharmatose DCL 14) | 29.14 |
| Prosolv 50 | 29.14 |
| Ac-Di-Sol (5%) | 3.75 |
| SDS (2%) | 1.5 |
| Aerosil (1%) | 0.75 |
| Magnesium stearate (1%) | 0.75 |
| Tablet weight | 75.03 |

### Example 4

### Enteric Polymer Coated Loratadine Tablet

IR tablets from lot # 1 (see Example 2) were used to manufacture enteric coated tablets. IR tablets were coated with Acryl-EZE MP 93018508 white (Colorcon Asia) using aqueous coating technique. A seal coat of Opadry YS-1-7006 clear (Colorcon Asia) was applied prior to Acryl-EZE coat. tion was formed.

IR tablets were coated first with a seal coating solution to achieve 3% weight gain and then with Acryl-EZE solution to achieve 23% and 30% weight gain. Dissolution profile of 23% Acryl-EZE coated Loratadine dissolution is given below.

TABLE 7
dissolution of Enteric coated tablets coated with 23% Acryl-EZE

| | % Loratadine released | | | | | | |
|---|---|---|---|---|---|---|---|
| | Time points | | | | | | |
| Media | (hours) | Tablet 1 | Tablet 2 | Tablet 3 | Tablet 4 | Tablet 5 | Tablet 6 |
| 0.1N HCl | 0 | 0 | 0 | 0 | 0 | 0 | 0 |
| | 2 | 0 | 0 | 0 | 0 | 0 | 0 |
| pH 6.8 | 2.5 | 72 | 69 | 82 | 90 | 81 | 80 |
| phosphate | 3 | 83 | 82 | 89 | 95 | 87 | 87 |
| buffer with 1.0% | 4 | 89 | 91 | 91 | 98 | 89 | 91 |
| SDS | 5 | 103 | 104 | 104 | 107 | 102 | 107 |

### Example 5

### Delayed Release Polymer Coated Loratadine Tablet

IR tablets were prepared as described in Example 2 (Lot #1). IR tablets were coated with Eudragit® L100 using solvent coating technique. A seal coat of Opadry YS-1-7006 clear (Colorcon Asia) was applied prior to Eudragit® L-100 coat. Lot #1 tablets were coated first with a seal coating solution to achieve 3% weight gain and then with Eudragit® L100 solution to achieve 22% and 30% weight gains. Coated tablets were cured for 2 hours at 40° C. Dissolution profile of delayed release Loratadine tablets coated with 30% Eudragit® L100 is presented below.

TABLE 8
Dissolution data of 30% Eudragit L100 coated Loratadine tablets

| | Time points | % Loratadine Released | | | | | |
|---|---|---|---|---|---|---|---|
| Media | (hours) | Tablet 1 | Tablet 2 | Tablet 3 | Tablet 4 | Tablet 5 | Tablet 6 |
| 0.1N HCl | 0 | 0 | 0 | 0 | 0 | 0 | 0 |
| | 2 | 0 | 0 | 0 | 0 | 0 | 0 |
| pH: 6.8 | 2.5 | 0 | 0 | 0 | 0 | 0 | 0 |
| phosphate | 3 | 2 | 19 | 53 | 2 | 51 | 1 |
| buffer with 1.0% | 4 | 82 | 100 | 89 | 97 | 103 | 103 |
| SDS | | | | | | | |

### Example 6

### Chlorpheniramine Maleate Coated Loratadine Tablet

An IR Chlorpheniramine maleate coated delayed release Loratadine dosage form can be prepared by spraying aqueous solution of Chlorpheniramine maleate onto either enteric coated Loratadine tablets (see Example 4) or delayed release Loratadine tablets (see Example 5). A seal coat of Opadry YS-1-7006 clear is preferred between a functional polymer coat and a Chlorpheniramine maleate coat to prevent any interaction between drug and polymer.

The coating solution was prepared as follows: Chlorpheniramine maleate, Polyethylene glycol 8000 and PVP K30 were dissolved in water. The mixture was stirred until clear solution was obtained. Talc and titanium dioxide were suspended in water to form a slurry which was passed through # 100 (150 micron) sieve and added to the Chlorpheniramine maleate solution described above. The seal coated enteric or delayed release Loratadine tablets were coated in the coating pan (Ganscoater®) using inlet set temperature at 55-60° C. with product bed temperature not more than 40° C. The composition of coating solution is given in the Table 9. The final dosage form contained 6 mg of Chlorpheniramine maleate and 10 mg of Loratadine.

TABLE 9
Chloropheniramine maleate coating solution composition (12,000
tablets)

| Ingredient | Amount per Batch (g) |
|---|---|
| Chloropheniramine maleate | 86.4 |
| Polyvinyl pyrrolidone (PVP K30 | 120 |
| Polyethylene glycol 8000 | 60 |
| Talc | 100 |
| Titanium Dioxide | 100 |
| DM water | 1533.6 |

We claim:

1. A method for improving the effectiveness of formulation containing a non-sedating or selective antihistamine, comprising co-administering a single dose of a sedating or non-selective antihistamine with a non-sedating or selective antihistamine.

2. The method of claim 1 wherein the non-sedating or selective antihistamine is selected from the group consisting of evocetrizine dihydrochloride, fexofenadine, loratadine, descarboethoxyloratadine, norastemizole, desmethylastemizole, cetirizine, acrivastine, ketotifen, temelastine, ebastine, epinastine, mizolastine, and setastine.

3. The method of claim 2 wherein the non-sedating or selective antihistamine is selected from the group consisting of loratadine and fexophenidine.

4. The method of claim 3 wherein the non-sedating or selective antihistamine is loratadine in a dosage between 2 and 80 mg, more preferably between 5 and 40 mg

5. The method of claim 1 wherein the sedating or non-selective antihistamine is selected from the group consisting of brompheniramine, chlorpheniramine, dexbrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, and hydroxyzine.

6. The method of claim 5 wherein the sedating or non-selective antihistamine is Chlorpheniramine in a dosage between 2 and 70 mg, more preferably between 4 and 24 mg per day

7. The method of claim 1 wherein the non-sedating or selective antihistamine is loratadine in a dosage of 10 mg and the sedating or non-selective antihistamine is chlorpheniramine in a dosage of 4 mg.

8. The method of claim 1 wherein the non-sedating or selective antihistamine is loratadine in a dosage of 10 mg and the sedating or non-selective antihistamine is chlorpheniramine in a dosage of 6 mg.

9. The method of claim 1 wherein the combination provides greater relief after 24 hours than administration of the non-sedating or selective antihistamine alone.

10. The method of claim 1 wherein a sedating or non-selective antihistamine is in an immediate release formulation and a non-sedating or selective antihistamine is in a delayed release formulation.

11. The method of claim 1 wherein the condition to be treated is one of allergic rhinitis and allergic eczema.

12. The method of claim 1 wherein the formulation is administered at bedtime.

13. A formulation for use in the method of claim 1.

14. The formulation of claim 13 in combination with packaging material describing the advantage of administering the two types of antihistamine together at bedtime to maximize symptom relief while minimizing next day drowsiness.
